(19) United States
(12) Patent Application Publication
(10) Pub. No.: US 2011/0236496 A1
(43) Pub. Date: Sep. 29, 2011

(54) Emulsions for Microencapsulation Comprising Biodegradable Surface-Active Block Copolymers as Stabilizers

(76) Inventor: Peter Markland, Birmingham, AL (US)

(21) Appl. No.: 13/071,633

(22) Filed: Mar. 25, 2011

Related U.S. Application Data
(60) Provisional application No. 61/317,738, filed on Mar. 26, 2010.

(51) Int. Cl. A61K 9/16

(52) U.S. Cl. 424/497

(57) ABSTRACT

Disclosed herein are surface-active biodegradable block copolymers comprising one or more hydrophobic blocks and one or more hydrophilic blocks. The surface-active polymers are used as stabilizers in emulsions which are used in microencapsulation processes. Also disclosed are microparticles prepared from the emulsions.

### CROSS-REFERENCE TO RELATED APPLICATIONS

This application claims the benefit of priority from prior U.S. Provisional Application No. 61/317,738, filed Mar. 26, 2010, the entire contents of which are incorporated herein by reference.

### BACKGROUND

Emulsion or double-emulsion processes are commonly used to form microparticles comprising a polymer matrix and an bioactive agent which is encapsulated within the polymer matrix. Such microparticles are useful for releasing the bioactive agent into a surrounding medium and are commonly used in drug-delivery, cosmetic, and agricultural applications. In a typical emulsion process, an emulsion is formed that comprises an encapsulating polymer and an bioactive agent, which are typically present in two distinct phases. Oftentimes, a dispersed phase is formed in a continuous phase using two or more immiscible liquids.

To stabilize emulsions for microencapsulation, a surfactant or surface-active agent is often used. Common surfactants include poly(vinyl alcohol), TWEEN, and others. Surfactants such as these can be difficult to completely remove from the final microparticle composition. Residual non-biodegradable surfactants can be disadvantageous for use in certain applications, such as drug-delivery.

### SUMMARY

Disclosed herein are surface-active, biodegradable, block copolymers comprising one or more hydrophobic blocks and one or more hydrophilic blocks. The surface-active polymers are used as stabilizers in emulsions which are used in microencapsulation processes. Also disclosed are microparticles prepared from the emulsions.

The advantages of the invention will be set forth in part in the description which follows, and in part will be obvious from the description, or may be learned by practice of the aspects described below. The advantages described below will be realized and attained by means of the elements and combinations particularly pointed out in the appended claims. It is to be understood that both the foregoing general description and the following detailed description are exemplary and explanatory only and are not restrictive.

### BRIEF DESCRIPTION OF THE DRAWINGS

FIG. 1 is a plot of goserelin release from the microparticles of Example 1.

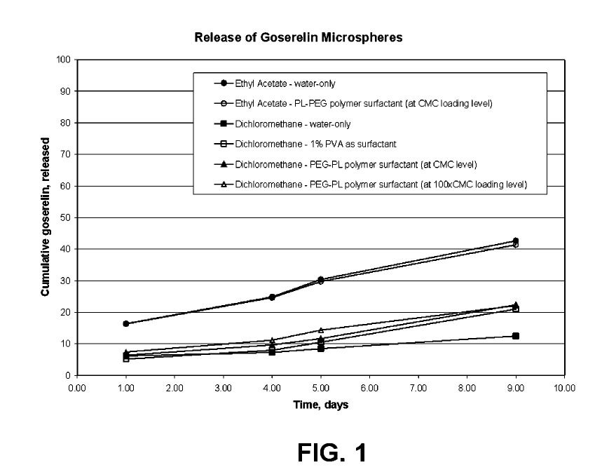

### DETAILED DESCRIPTION

Before the present compounds, compositions, composites, articles, devices and/or methods are disclosed and described, it is to be understood that the aspects described below are not limited to specific compounds, compositions, composites, articles, devices, methods, or uses as such may, of course, vary. It is also to be understood that the terminology used herein is for the purpose of describing particular aspects only and is not intended to be limiting.

In this specification and in the claims that follow, reference will be made to a number of terms that shall be defined to have the following meanings:

Throughout this specification, unless the context requires otherwise, the word “comprise,” or variations such as “comprises” or “comprising,” will be understood to imply the inclusion of a stated integer or step or group of integers or steps but not the exclusion of any other integer or step or group of integers or steps.

It must be noted that, as used in the specification and the appended claims, the singular forms “a,” “an” and “the” include plural referents unless the context clearly dictates otherwise. Thus, for example, reference to “a bioactive agent” includes mixtures of two or more such agents, and the like.

“Optional” or “optionally” means that the subsequently described event or circumstance can or cannot occur, and that the description includes instances where the event or circumstance occurs and instances where it does not.

Ranges may be expressed herein as from “about” one particular value, and/or to “about” another particular value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint.

The term “microparticle” is used herein to refer generally to a variety of structures having sizes from about 10 nm to 2000 microns (2 millimeters) and includes microcapsule, microsphere, nanoparticle, nanocapsule, nanosphere as well as particles, in general, that are less than about 2000 microns (2 millimeters). In one aspect, the bioactive agent is encapsulated in the microparticle.

“Biodegradable” is generally referred to herein as a material that will erode to soluble species or that will degrade under physiologic conditions to smaller units or chemical species that are, themselves, non-toxic (biocompatible) to the subject and capable of being metabolized, eliminated, or excreted by the subject.

A “bioactive agent” refers to an agent that has biological activity. The biological agent can be used to treat, diagnose, cure, mitigate, prevent (i.e., prophylactically), ameliorate, modulate, or have an otherwise favorable effect on a disease, disorder, infection, and the like. A “releasable bioactive agent” is one that can be released from a disclosed microparticle. Bioactive agents also include those substances which affect the structure or function of a subject, or a pro-drug, which becomes bioactive or more bioactive after it has been placed in a predetermined physiological environment.

Disclosed are compounds, compositions, and components that can be used for, can be used in conjunction with, can be used in preparation for, or are products of the disclosed methods and compositions. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if a number of different polymers and agents are disclosed and discussed, each and every combination and permutation of the polymer and agent are specifically contemplated unless specifically indicated to the contrary. Thus, if a class of molecules A, B, and C are disclosed as well as a class of molecules D, E, and F and an example of a combination molecule, A-D is disclosed, then even if each is not individually recited, each is individually and collectively contemplated. Thus, in this example, each of the combinations A-E, A-F, B-D, B-E, B-F, C-D, C-E, and C-F are specifically contemplated and should be considered disclosed from disclosure of A, B, and C; D, E, and F; and the example combination A-D. Likewise, any subset or combination of these is also specifically contemplated and disclosed. Thus, for example, the sub-group of A-E, B-F, and C-E are specifically contemplated and should be considered disclosed from disclosure of A, B, and C; D, E, and F; and the example combination A-D. This concept applies to all aspects of this disclosure including, but not limited to, steps in methods of making and using the disclosed compositions. Thus, if there are a variety of additional steps that can be performed it is understood that each of these additional steps can be performed with any specific embodiment or combination of embodiments of the disclosed methods, and that each such combination is specifically contemplated and should be considered disclosed.

The present invention provides for emulsions, processes, and microparticles prepared from the emulsions and processes that include a biodegradable surface-active block copolymer that comprises one or more hydrophilic blocks and one or more hydrophobic blocks. The surface-active block copolymer can aid in stabilizing the emulsion and can result in comparable and even improved encapsulation efficiencies, relative to traditional emulsion processes that primarily utilize non-biodegradable surfactants, such as poly(vinyl alcohol), TWEEN, and the like. The use of the hydrophobic block copolymers in emulsions can also avoid disadvantages typically associated with the use of non-biodegradable surfactants, particularly for pharmaceutical applications. In addition, blocks of the surface-active block copolymer can be chosen to provide a beneficial environment for the bioactive agent within the interior of the microparticle; for example a hydrophilic polysaccharide or poly(ethylene glycol) block may preferentially accumulate into the aqueous bioactive agent-containing phase of a double-emulsion process and thereby concentrate around the bioactive agent which, in some instances, may provide beneficial effects in the final microparticle product (such as hydrophilicity, bioactive agent-polymer interactions, bioactive agent stability, etc).

The emulsions of the invention generally comprise an outer continuous phase of a first liquid and an inner dispersed phase of a second liquid that is at least partially immiscible with the first liquid; and wherein the emulsion comprises: (a) a biodegradable surface-active block copolymer that comprises one or more hydrophilic blocks and one or more hydrophobic blocks; (b) a biodegradable encapsulating polymer; and (c) an bioactive agent present in the inner dispersed phase.

The emulsions of the invention can, in some examples, be substantially free of non-biodegradable polymers or surfactants, such as poly(vinyl alcohol), TWEEN, etc., e.g., can contain 0.1% or less, including an emulsion that is completely free of non-biodegradable polymers or surfactants. In other aspects, non-biodegradable polymers or surfactants can be present, for example in an amount up to 1%, e.g., from greater than 0.1% to 1%.

In one aspect, the surface-active block copolymer is at least partially water soluble and is mixed with the other emulsion components as an aqueous solution. In some examples, the emulsion is either an oil-in-water O/W emulsion, a solid-in-oil-in water emulsion, or a water-in-oil-in-water (W/O/W) double emulsion. In an oil-in-water emulsion, the surface-active block copolymer will primarily be present in the outer continuous phase, which is the aqueous phase.

A solid-in-oil-in-water emulsion can be useful for encapsulating a solid bioactive agent that is insoluble or somewhat insoluble in the organic solvent used in the oil phase. The solid bioactive agent can be dispersed in the organic phase, and the biodegradable encapsulating polymer can be present in the oil phase surrounding the solid bioactive agent (the organic phase). The surface-active block copolymer will preferably be present in the outer continuous phase of such an emulsion, i.e., the outer aqueous phase.

In a water-in-oil-in-water (W/O/W) double emulsion, the bioactive agent will typically be present in the inner aqueous phase and surrounded by the oil phase that comprises an organic solvent and the biodegradable encapsulating polymer dissolved in the organic solvent. In this example, the inner dispersed phase comprises the inner W/O phases, which is dispersed in the outer aqueous continuous phase. In this double-emulsion, the surface-active block copolymer can be present in either aqueous phases, i.e., the aqueous phase of the inner dispersion (also called the primary emulsion) or the outer aqueous continuous phase. In one preferred aspect, the surface-active block copolymer is present in the aqueous phase of the inner dispersion (the primary emulsion) thereby serving as an emulsion stabilizer for the inner W/O emulsion.

In some examples, the surface-active block copolymer may be present in more than one phase, due to its hydrophobic/hydrophilic character. Typically, the hydrophilic region of the block copolymer will orient toward or be present within an aqueous phase, while the hydrophobic region of the block copolymer will orient or be present within an organic phase. This hydrophobic/hydrophilic character of the block copolymer gives rise to surfactant like properties and can thereby allow the emulsions of the invention to be stabilized.

The amount of the surface-active block copolymer in the emulsion will vary. The amount of the surface-active block copolymer in the emulsion will depend on the amount of block copolymer in the starting solution or dispersion used to prepare the emulsion. For example, in the oil-in-water, solid-in-oil-in-water, or water-in-oil-in-water emulsions discussed above, the biodegradable surface-active block copolymer is mixed with the one or more immiscible phases as an aqueous solution. Such an aqueous solution can also comprise the bioactive agent, for example when preparing the water-in-oil primary emulsion of a double emulsion.

Generally, the amount of surface-active block copolymer in the aqueous solution can range from 0.001 mg/L to 10 g/L, preferably from 0.001 mg/L to 200 mg/L, and more preferably 0.001 mg/L to 100 mg/L, depending on the solubility of the surface-active block copolymer. Preferably, the surface-active block copolymer has a solubility of at least 0.1 mg/L in water. In further examples, the amount of surface-active block copolymer in the aqueous solution ranges from 0.1 mg/L to 100 mg/L, 1 mg/L to 100 mg/L, or 10 mg/L to 100 mg/L.

The amount of the surface-active block copolymer that is present in a particular phase of the emulsion may vary from the amounts discussed above after mixing the immiscible phases and forming the emulsion. However, it is generally contemplated that in an oil-in-water emulsion, a solid-in-oil-in-water emulsion, or a water-in-oil-in-water emulsion, the surface-active block copolymer can be present in an amount ranging from 0.001 mg/L of the aqueous phase to 10 g/L of the aqueous phase, including any of the specific concentrations listed above. In a water-in-oil-in-water emulsion, the surface-active block copolymer can be present in the inner aqueous phase of the inner dispersed phases or the outer aqueous continuous phase in any of the amounts discussed.

It is preferable that most, if not all, of the bioactive agent that is to be encapsulated by the biodegradable encapsulating polymer will be present in the inner phase. In the example of a double emulsion, the bioactive agent will be present in the inner aqueous phase of the inner dispersed phases (the primary emulsion). As discussed above, the “inner dispersed phase” can itself include more than one phase, for example in the double-emulsions discussed above that comprises an inner phase that comprises an inner aqueous phase and an organic phase surrounding the inner aqueous phase. Together, this water-in-oil primary emulsion constitutes the inner dispersed phase, which is the primary emulsion.

In a single emulsion, which can be either an oil-in-water or water-in-oil emulsion, the bioactive agent can be present in an amount of from 1% to 75% by weight of the inner dispersed oil phase or the inner dispersed aqueous phases, including without limitation, amounts of about 5%, 10%, 15%, 20%, 30%, 40%, 50%, 60%, or 70% by weight bioactive agent. In a double-emulsion, the bioactive agent will typically be present in the inner organic or aqueous phase of the inner primary emulsion in an amount of from 1% to 70% by weight of the inner organic or aqueous phase of the inner primary emulsion, including without limitation, amounts of about 5%, 10%, 15%, 20%, 30%, 40%, 50%, 60%, or 70% by weight bioactive agent. In an emulsion wherein the bioactive agent is insoluble or partially soluble (i.e. an emulsion comprising a solid-in-liquid component), the bioactive agent can be present in any of the amounts discussed above which generally range from 1% to 70% by weight of bioactive agent relative to the total weight of the bioactive agent and the phase in which the bioactive agent is dispersed.

“Theoretical loading” of the bioactive agent is calculated from the weight of the bioactive agent relative to the total combined weight of solids that are used to form the microparticles (this is often the combined weights of the bioactive agent and biodegradable polymer). The weight of the liquid or solvent (or inner aqueous phase) is not included in the theoretical loading calculation. As an example, a process using a ratio of 5 grams of bioactive agent and 15 grams of biodegradable polymer would have a theoretical loading of 25%, regardless of how much solvent was used to prepare the dispersed phase solutions or dispersions. The “actual loading” divided by the “theoretical loading” is referred to herein as the “encapsulation efficiency” (expressed as a %). Actual loading can be determined by HPLC determinations of the total amount of bioactive agent encapsulated in a given amount of the microparticle product by extracting the bioactive agent from the microparticle product and quantifying the amount using HPLC (with actual loading expressed as a wt % bioactive agent).

The biodegradable encapsulating polymer is preferably a polymer that is at least partially soluble in an organic solvent and slightly soluble to insoluble in water. Generally, the biodegradable encapsulating polymer is dissolved in an organic solvent in a concentration in an organic phase of the emulsion of from 0.01 to 90% by weight, preferably 0.1 to 80% by weight, including without limitation, about 5%, 10%, 15%, 20%, 30%, 40%, 50%, 60%, 70%, or 80% by weight.

A single O/W or W/O type emulsion is prepared by mixing a solution of the biodegradable encapsulating polymer in an outer continuous phase solvent (either water or organic) with an inner phase solvent comprising the bioactive agent and then emulsifying the mixture. As discussed above, the surface-active block copolymer can be present in either the inner phase or outer phase. A double O/W/O or W/O/W emulsion is prepared by emulsifying a single primary emulsion with an outer continuous phase solvent, similar to the preparation of the primary emulsion.

The emulsifying procedures can be carried out by a conventional method, for example, by mixing the two or more immiscible phases with stirring by using a known emulsifying apparatus such as a propeller stirrer, a turbine impeller mixer, a high-pressure emulsifier, an ultrasonic dispersion mixer, a static mixer, a packed bed column (e.g., a FormEZE column), and the like. The emulsification may also be done by other methods such as a membrane emulsifying method, a spraying method, among other methods.

The emulsification by a membrane emulsifying method can be carried out by providing a porous membrane (e.g., porous ceramics which surface is optionally chemically modified, porous glass, etc.) between two immiscible phases and extruding one of the phases into the other phase through fine holes of the porous membrane under pressure, and if desired with stirring of the one or more phases. Such a method is discussed in more detail in the Journal of Microencapsulation, vol. 11 (2), pp. 171-178, 1994.

The emulsification by a spraying method can be carried out by spraying the one phase onto the other phase with a conventional spraying apparatus. The spraying apparatus includes, for example, an air nozzle, a pressure nozzle, an ultrasonic nozzle, a rotary atomizer, and the like.

The emulsions of the invention thus will generally contain the inner dispersed phase and the outer continuous phase wherein the outer continuous phase is in the ratio of 1 to 10,000 parts by volume, preferably 2 to 1,000 parts by volume, per 1 part by volume of the inner dispersed phase.

The aqueous phase of any of the disclosed emulsions can comprise any suitable aqueous solvent. One non-limiting example of an aqueous solvent is water. In one aspect, water can be mixed with another miscible solvent, for example, ethanol, methanol, DMSO, DMF, isopropyl alcohol, among many other water-miscible polar solvents. In various aspects, the first phase can contain other compounds, such as buffers, salts, sugars, and/or viscosity-modifying agents, or combinations thereof.

The organic phase of the emulsions typically comprises an organic solvent having a boiling point lower than that of water including halogenated aliphatic hydrocarbon solvents (e.g., methylene chloride, chloroform, carbon tetrachloride, chloroethane, dichloroethane, trichloroethane, etc.), alkyl ester solvents (e.g., methyl acetate, ethyl acetate, etc.), aromatic hydrocarbon solvents (e.g., benzene), aliphatic hydrocarbon solvents (e.g., n-hexane, n-pentane, cyclohexane, etc.), ketone solvents (e.g., methyl ethyl ketone, etc.), and ether solvents (e.g., diethyl ether, diisopropyl ether, methyl isobutyl ether, methyl tert-butyl ether, tetrahydrofuran, etc.). In some aspects, the organic solvent is a polymer which can dissolve the biodegradable encapsulating polymer.

The organic solvents have preferably a boiling point of 15-60° C. lower than that of water under the condition of removal of the organic solvents. Particularly preferred organic solvents are methylene chloride, chloroform, and ethyl acetate.

Once the emulsion is formed, microparticles can be formed from the emulsion. The microparticles are typically formed by removing the solvent for the biodegradable encapsulating polymer (e.g. the organic solvent). The solvent for the biodegradable encapsulating polymer can be removed by any suitable methods. In one aspect, the solvent can be removed by extracting the solvent with an extraction liquid, such as water. In other aspects, the solvent can be removed by drying, such as by spray drying, drying under reduced pressure, solvent evaporation, lyophilization, or a combination thereof.

Emulsion methods for preparing microparticles are further discussed in Jeffery, et al., “The preparation and characterisation of poly(lactide-co-glycolide) microparticles. I: Oil-In-water emulsion solvent evaporation,” Int. J. Pharm. 77(2-3):169-175 (1991); Jeffery, et al., “The Preparation and Characterization of Poly(lactide-co-glycolide) Microparticles. II. The Entrapment of a Model Protein using a (Water-in-Oil)-in-Water Emulsion Solvent Evaporation Technique,” Pharm. Res. 10(3):362-368 (1993). Solvent evaporation methods are discussed Wichert, B. and Rohdewald, P. (1993) J. Microencapsul. 10:195.

The surface active biodegradable polymer can comprise a variety of hydrophobic and hydrophilic blocks and can generally be an AB copolymer, an ABA tri block copolymer, BAB tri block copolymer, an (AB)ₙ multiblock copolymer, a graft copolymer, a star block copolymer, or a dendrimer.

The molecular weight of the individual hydrophobic and hydrophilic blocks as well as the molecular weight of the polymer as a whole can vary. The molecular weight of the one or more hydrophilic blocks of the surface-active block copolymer can be from 250 to 20,000 Daltons (Da), from 500 to 8,000 Daltons (Da), or from 1,000 to 6,000 Daltons (Da). The molecular weight of the one or more hydrophobic blocks of the surface-active block copolymer can be from 250 to 20,000 Daltons (Da), from 500 to 8,000 Daltons (Da), or from 1,000 to 6,000 Daltons (Da).

The surface-active block copolymer can have a molecular weight of from 500 to 75,000 Daltons (Da), 500 to 75,000 Da, or 500 to 25,000 Da e.g., 1,000 to 15,000 Daltons (Da), or 2,000 to 10,000 Daltons (Da). A linear block copolymer (AB, ABA, etc.) can have any of these molecular weights. The molecular weight of such a linear polymer can be determined by gel-permeation chromatography (GPC). For branched polymers, such as dendrimers or graft copolymers, molecular weights can be higher, for example, from 500 to 100,000 Da, 500 to 25,000, e.g., 1,000 to 15,000 Daltons (Da), or 2,000 to 10,000 Daltons (Da). For branched polymers, such as dendrimers, graft copolymers, and the like, molecular weights are preferably measured using methods that determine absolute molecular weight, such as light scattering.

In one aspect, the biodegradable surface-active block copolymer is nonionic, i.e., the polymer contains no charged residues.

Examples of the surface-active block copolymer include polymers that comprise one or more hydrophilic blocks selected from poly(ethylene glycol) (PEG), and poly(vinylpyrrolidone) (PVP), and polysaccharides such as dextrin, starch, dextran, hyaluronic acid, cellulose, including modified versions of cellulose, such as methylcellulose; and one or more hydrophobic blocks selected from poly(lactide), poly(glycolide), poly(caprolactone), poly(valerolactone), poly(hydroxybutyrate), and copolymers thereof.

The microparticles of the invention, which can be formed from the emulsions will generally comprise the biodegradable encapsulating polymer as the polymer matrix and the bioactive agent encapsulated in the polymer matrix. The microparticles can also have residual surface-active block copolymer encapsulated in the matrix. Generally, the microparticles can have a range of mean particle sizes, for example, from 5 microns to 150 microns, or from 10 microns to 80 microns, or from 30 microns to 80 microns.

The encapsulating polymers are typically prepared as a solution or dispersion of polymer in an organic solvent and then mixed with an aqueous solution or dispersion of bioactive agent to form an emulsion, as discussed above. Suitable biodegradable polymers for use with the invention include without limitation poly(lactide), a poly(glycolide), a poly(lactide-co-glycolide), a poly(caprolactone), a poly(orthoester), a poly(phosphazene), a poly(hydroxybutyrate) a copolymer containing a poly(hydroxybutarate), a poly(lactide-co-caprolactone), a polycarbonate, a polyesteramide, a polyanhydride, a poly(dioxanone), a poly(alkylene alkylate), a copolymer of polyethylene glycol and a polyorthoester, a biodegradable polyurethane, a poly(amino acid), a polyamide, a polyesteramide, a polyetherester, a polyacetal, a polycyanoacrylate, a poly(oxyethylene)/poly(oxypropylene) copolymer, polyacetals, polyketals, polyphosphoesters, polyhydroxyvalerates or a copolymer containing a polyhydroxyvalerate, polyalkylene oxalates, polyalkylene succinates, poly(maleic acid), and copolymers, terpolymers, combinations thereof.

The biodegradable polymer can comprise one or more residues of lactic acid, glycolic acid, lactide, glycolide, caprolactone, hydroxybutyrate, hydroxyvalerates, dioxanones, polyethylene glycol (PEG), polyethylene oxide, or a combination thereof. More preferably, the hydrophobic polysaccharide derivative is blended with one or more polymers that comprise one or more residues of lactide, glycolide, caprolactone, or a combination thereof.

In some aspects, the biodegradable polymer comprises one or more lactide residues. The polymer can comprise any lactide residue, including all racemic and stereospecific forms of lactide, including, but not limited to, L-lactide, D-lactide, and D,L-lactide, or a mixture thereof. Useful polymers comprising lactide include, but are not limited to poly(L-lactide), poly(D-lactide), and poly(DL-lactide); and poly(lactide-co-glycolide), including poly(L-lactide-co-glycolide), poly(D-lactide-co-glycolide), and poly(DL-lactide-co-glycolide); or copolymers, terpolymers, combinations, or blends thereof. Lactide/glycolide polymers can be conveniently made by melt polymerization through ring opening of lactide and glycolide monomers. Additionally, racemic DL-lactide, L-lactide, and D-lactide polymers are commercially available. The L-polymers are more crystalline and resorb slower than DL-polymers. In addition to copolymers comprising glycolide and DL-lactide or L-lactide, copolymers of L-lactide and DL-lactide are commercially available. Homopolymers of lactide or glycolide are also commercially available.

When poly(lactide-co-glycolide), poly(lactide), or poly(glycolide) is used, the amount of lactide and glycolide in the polymer can vary. For example, the biodegradable polymer can contain 0 to 100 mole %, 40 to 100 mole %, 50 to 100 mole %, 60 to 100 mole %, 70 to 100 mole %, or 80 to 100 mole % lactide and from 0 to 100 mole %, 0 to 60 mole %, 10 to 40 mole %, 20 to 40 mole %, or 30 to 40 mole % glycolide, wherein the amount of lactide and glycolide is 100 mole %. In a further aspect, the biodegradable polymer can be poly(lactide), 95:5 poly(lactide-co-glycolide) 85:15 poly(lactide-co-glycolide), 75:25 poly(lactide-co-glycolide), 65:35 poly(lactide-co-glycolide), or 50:50 poly(lactide-co-glycolide), where the ratios are mole ratios.

In a further aspect, the biodegradable polymer can comprise a poly(caprolactone) or a poly(lactide-co-caprolactone). For example, the polymer can be a poly(lactide-caprolactone), which, in various aspects, can be 95:5 poly(lactide-co-caprolactone), 85:15 poly(lactide-co-caprolactone), 75:25 poly(lactide-co-caprolactone), 65:35 poly(lactide-co-caprolactone), or 50:50 poly(lactide-co-caprolactone), where the ratios are mole ratios.

In some aspects, both the surface-active block copolymer and the encapsulating polymer comprise the same or similar biodegradable residues. However, it is understood that the surface-active block copolymer and the encapsulating polymer are separate polymers, i.e., they are not bonded together.

A variety of bioactive agent can be used with the disclosed processes and can be present in the emulsions and encapsulated within the microparticles of the invention. The bioactive agent can include cosmetics and agricultural products. and bioactive agents. In one aspect, the bioactive agent comprises a water soluble V. Examples suitable water soluble bioactive agents or drugs include without limitation peptides, proteins, aptamers, nucleic acids, RNA, DNA, and RNAi complexes such as RNA-transfection complexes including siRNA transfection complexes.

The bioactive agent will typically be present in the inner dispersed phase of the emulsion and can be present in any suitable amount. In some examples, the bioactive agent is present in the inner dispersed phase in an amount of from 1% to 75% by weight (e.g., 1% to 50%, 1% to 30%, 1% to 20%, or 1% to 10%) of the dispersed phase, or by weight of the original formulation used to prepare the dispersed phase, such as an aqueous solution or aqueous dispersion of the bioactive agent which is to be dispersed in the continuous phase, or which is used to prepare the primary emulsion of a double-emulsion.

Various forms of bioactive agents can be used, which are capable of being released from the microparticle into a subject. A liquid or solid bioactive agent can be incorporated into the microparticles described herein. The bioactive agents can be water soluble or water-insoluble. In some aspects, the bioactive agent is at least very slightly water soluble, and preferably moderately water soluble. The bioactive agents can include salts of the active ingredient. As such, the bioactive agents can be acidic, basic, or amphoteric salts. They can be nonionic molecules, polar molecules, or molecular complexes capable of hydrogen bonding. The bioactive agent can be included in the devices in the form of, for example, an uncharged molecule, a molecular complex, a salt, an ether, an ester, an amide, polymer drug conjugate, or other form to provide the effective biological or physiological activity.

### EXAMPLES

The following examples are put forth so as to provide those of ordinary skill in the art with a complete disclosure and description of how the compounds, compositions, articles, devices and/or methods claimed herein are made and evaluated, and are intended to be purely exemplary of the invention and are not intended to limit the scope of what the inventors regard as their invention. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### Example 1

Microparticle formulations containing goserelin acetate were prepared using a double-emulsion, solvent-extraction microencapsulation process as described below. Formulations were prepared using dissolved goserelin in aqueous solution which was dispersed in the dispersed phase (DP) solution thereby forming the primary emulsion of the double-emulsion process. For all microparticle formulations, a biodegradable polymer 50:50 poly(DL-lactide-co-glycolide) (Lakeshore Biomaterials brand, SurModics Pharmaceuticals, Birmingham, Ala.) was used to prepare the DP solution.

First, four batches were prepared using dichloromethane as the organic solvent for the polymer solution of the DP (Table 1). For Batch 00277-058 block copolymer of PEG-poly(DL-lactide) (PEG-PL) was used as a polymer surfactant in the aqueous phase of the primary emulsion. In this example, the polymer surfactant solution was prepared by dissolving 20 mg of a 100 DLmPEG5K2CE polymer (SurModics Pharmaceuticals, Birmingham, Ala.) in 1 L of deionized water. A drug solution was prepared by dissolving 200 mg of goserelin acetate (Genzyme pharmaceuticals) in 1 mL of the polymer surfactant solution. A dispersed phase (DP) solution was prepared by dispersing the drug solution into 12 g of polymer solution consisting of 15 wt % 50:50 poly(DL-lactide-co-glycolide) (0.45 dug) in methylene chloride. An IKA ultra-turrax probe mixer was used to disperse the drug solution into the polymer solution thereby forming the DP solution in the form of a primary emulsion. The resulting DP solution was emulsified into 200 g of a continuous phase (CP) solution consisting of 2 wt % aqueous polyvinyl alcohol (PVA) and containing 1.6 wt % methylene chloride. Emulsification of the DP and CP was performed in a discontinuous manner by introducing the DP into the CP solution which was stirred using a Silverson L4R-T Probe mixer and high shear screen (speed setting 900 rpm). Microparticles were prepared by mixing the emulsion for 30 sec then pouring directly into 2600 g of stirred deionized water. The resulting suspension was stirred for 30 min after which time the microparticle product was isolated by screening through 125 and 20 micron test sieves. The microparticles collected on the 20-micron sieve were washed with 2 L of de-ionized water. After washing the microparticles were allowed to dry on the 20 micron sieve in a laminar flow hood.

For Batch 00277-067 block copolymer of PEG-PL was also used as a polymer surfactant in the aqueous phase of the primary emulsion; however in this example the polymer surfactant was used at a 100-fold higher concentration than was used for batch 00277-058. In this example, the polymer surfactant solution was prepared by dissolving 20 mg of a 100 DLmPEG5K2CE polymer (Lakeshore Biomaterials) in 10 mL of deionized water. A drug solution was prepared by dissolving 200 mg of goserelin acetate (Genzyme pharmaceuticals) in 1 mL of the polymer surfactant solution. Microparticles were prepared as described for batch 00277-058.

For batch 00277-064, 1% PVA was used as the polymer surfactant solution instead of the dilute PEG-PL block copolymer. All other conditions were the same as for Batch 00277-058.

For Batch 00277-061, deionized water used in place of a surfactant solution. All other conditions were the same as for Batch 00277-058.

Next, two batches were prepared using ethyl acetate as the organic solvent to prepare the polymer solution of the DP and for saturation of the CP solution (Table 2). For batch 00277-113 was prepared using the same concentration of the PEG-PL polymer surfactant as was used in batch 00277-058 (20 mg/L). As mentioned, ethyl acetate was used to prepare a 15 wt % polymer solution and then ethyl acetate was added to the CP solution (instead of dichloromethane) at a saturating level of about 7.5 wt %. All other conditions were the same as was used to prepare batch 00277-058. As a control, batch 00277-109 was prepared using only water to dissolve the goserelin acetate and to prepare the primary emulsion (no polymer surfactant solution was used). Again, ethyl acetate was used instead of methylene chloride as described in batch 00277-113. All other processing conditions were the same as was used to prepare batch 00277-058.

Next, four batches were prepared using dichloromethane as the organic solvent to prepare the polymer solution of the DP and for saturation of the CP solution (Table 3). As is indicated in Table 3, batches were prepared using either the PEG-PL polymer surfactant solution (at either the 20 mg/L or the 20 mg/10 mL levels, as indicated) or pure water to dissolve the drug and for preparation of the primary emulsion. Each of these two sets of samples, then, were prepared using two different ratios of aqueous phase:organic phase to prepare the primary emulsion. In the first two samples (Batches 00277-117 and 00277-105), 100 mg goserelin acetate was dissolved in 1 mL of aqueous solution (either the PEG-PL polymer surfactant solution or deionized water). In contrast, the remaining two samples (batches 00277-129 and 00277-132 were prepared by dissolving 100 mg goserelin acetate in only 0.5 mL of aqueous solution (either the PEG-PL polymer surfactant solution or deionized water). All other processing conditions used to prepare these formulations were the same as was used to prepare batch 00277-058.

Microsphere formulations were analyzed for goserelin content. A 20 to 30 mg portion of each formulation was weighed into a 25 mL volumetric flask and 5 mL of glacial acetic acid was added. The sample was allowed to dissolve. After the entire sample had dissolved, the flask was diluted to volume with PBS. The mixture was filtered using a 0.45 μm syringe filter. The filtered solution was transferred to a HPLC vial and samples analyzed by HPLC (UV at 220 nm). Analysis was performed in triplicate. Controls were prepared by weighing drug and polymer and performing the same step as above.

In vitro release profiles were determined for the goserelin formulations. A 20-30 mg sample of each formulation was weighed into a 20 mL glass vial and 10 mL of PBS is added. The vial was placed in a shaking incubator whose temperature was maintained at 37° C. At the appropriate time point 9 mL of buffer was removed from the vial and 9 mL of fresh buffer is added back to the vial. Care is taken not remove any microspheres. The vial was placed back into the incubator until the next time point. The buffer containing released goserelin was assayed for goserelin using a HPLC method (UV at 220 nm). Analysis was performed in triplicate. Cumulative released goserelin was reported.

The results are shown in Tables 1-3.

TABLE 1
Microencapsulation of goserelin acetate using different primary emulsion
surfactants (polymer solution: 50:50 DL-PLG dissolved at 15 wt % in dichlormethane)

| Lot | | surfactant | Theoretical drug loading | Actual loading | Encapsulation | Mean particle size, |
|---|---|---|---|---|---|---|
| Number | surfactant | concentration | level, wt % | level, wt % | efficiency, % | microns |
| 00277- | PL-PEG | 20 mg/L | 10 | 9.1 | 91 | 69 |
| 058 | | | | | | |
| 00277- | PL-PEG | 2 mg/mL | 10 | 9.4 | 94 | 78 |
| 067 | | | | | | |
| 00277- | PVA | 1 wt % | 10 | 9.2 | 92 | 53 |
| 064 | | | | | | |
| 00277- | (none) | (none) | 10 | 8.6 | 86 | 60 |
| 061 | | | | | | |

TABLE 2
Microencapsulation of goserelin acetate in ethyl
acetate solvent (polymer solution: 50:50 DL-PLG
dissolved at 15 wt % in ethyl acetate)

| | | | Theoretical | Actual | Encap- |
|---|---|---|---|---|---|
| | | surfactant | drug | loading | sulation |
| Lot | | con- | loading | level, | efficiency, |
| Number | surfactant | centration | level, wt % | wt % | % |
| 00277-113 | PL-PEG | 20 mg/L | 10 | 9.0 | 90 |
| 00277-109 | (none) | (none) | 10 | 8.2 | 82 |

TABLE 3
Microencapsulation of goserelin acetate at different ratios of inner aqueous
phase during preparation of the primary emulsion (polymer solution: 50:50
DL-PLG dissolved at 15 wt % in dichlormethane)

| | | | volume | Theoretical | Actual | |
|---|---|---|---|---|---|---|
| | | | aqueous | drug | loading | |
| Lot | | Surfactant | phase, | loading | level, | Encapsulation |
| Number | Surfactant | concentration | mL | level, wt % | wt % | efficiency, % |
| 00277-117 | PL-PEG | 20 mg/L | 1 mL | 5 | 4.3 | 86 |
| 00277-105 | (none) | (none) | 1 mL | 5 | 4.0 | 80 |
| 00277-129 | PL-PEG | 2 mg/mL | 0.5 mL | 5 | 5.0 | 100 |
| 00277-132 | (none) | (none) | 0.5 mL | 5 | 4.2 | 84 |

### Example 2

The model protein bovine serum albumin (BSA) was used to prepare to batches listed in Table 4. Batch 00277-138 was prepared using 200 mg of BSA dissolved into 1 mL of the 2 mg/mL PEG-PL polymer surfactant solution. All other processing conditions used to prepare this formulation were the same as those used to prepare batch 00277-067. As a control, a second batch was prepared, batch 0277-135, in which 200 mg BSA was dissolved into deionized water; all other processing conditions were the same as those used to prepare batch 00277-058.

Microparticle formulations were analyzed for BSA content. 20 to 30 mg of each formulation was weighed into a 2 mL eppendorf tube and 1 mL of ethyl acetate is added. The microspheres were allowed to dissolve. The contents of the tube were centrifuge in a microcentrifuge at 14,000 rpm for 10 min. Care was taken not to remove any solid protein and approximately 800 uL of ethyl acetate is removed. 800 uL of fresh ethyl acetate is added and content of the tube is recentrifuged. After the ethyl acetate is removed. A flow of nitrogen (10 mL/min) is directed into to the tube to dry the remaining solvent. The tube is place on a freeze dryer to further dry off the solvent. After drying the contents of the tube is dissolved in 1 mL of PBS and transferred to a 10 mL. More PBS is added to the tube and transferred to the flask. The contents of the flask was diluted to volume with PBS. A portion was transferred to a HPLC vial and samples analyzed by HPLC (UV at 220 nm). Analysis was performed in triplicate. Controls were prepared by weighing drug and polymer and performing the same step as above.

The results are listed in Table 4.

TABLE 4
Microencapsulation of BSA using dichloromethane as a
processing solvent (polymer solution: 50:50 DL-PLG
dissolved at 15 wt% in dichloromethane)

| | | | Theoretical | Actual | Encap- |
|---|---|---|---|---|---|
| | | Surfactant | drug | loading | sulation |
| Lot | | con- | loading | level, | efficiency, |
| Number | Surfactant | centration | level, wt % | wt % | % |
| 00277-138 | PL-PEG | 2 mg/mL | 10 | 9.5 | 94 |
| 00277-135 | (none) | (none) | 10 | 7.9 | 79 |

Various modifications and variations can be made to the compounds, composites, kits, articles, devices, compositions, and methods described herein. Other aspects of the compounds, composites, kits, articles, devices, compositions, and methods described herein will be apparent from consideration of the specification and practice of the compounds, composites, kits, articles, devices, compositions, and methods disclosed herein. It is intended that the specification and examples be considered as exemplary.

What is claimed is:

1. An emulsion comprising an outer continuous phase of a first liquid and an inner dispersed phase of a second liquid that is at least partially immiscible with the first liquid; wherein the emulsion further comprises: (a) a biodegradable surface-active block copolymer that comprises one or more hydrophilic blocks and one or more hydrophobic blocks; (b) a biodegradable encapsulating polymer; and (c) a bioactive agent.

2. The emulsion of claim 1 wherein the inner dispersed phase or the outer continuous phase comprises at least 0.001 mg/L of the surface-active block copolymer.

3. The emulsion of claim 1 wherein the biodegradable surface-active block copolymer is nonionic.

4. The emulsion of claim 1 wherein the surface-active block copolymer comprises one or more hydrophilic blocks of poly(ethylene glycol) (PEG), poly(vinylpyrrolidone) (PVP), or a polysaccharide; and one or more hydrophobic blocks of poly(lactide), poly(glycolide), poly(caprolactone), poly(valerolactone), poly(hydroxybutyrate), or a copolymer thereof.

5. The emulsion of claim 1 wherein the one or more hydrophilic blocks of the surface-active block copolymer has a molecular weight of from 250 to 20,000 Daltons (Da).

6. The emulsion of claim 1 wherein the one or more hydrophobic blocks of the surface-active block copolymer has a molecular weight of from 250 to 20,000 Daltons (Da).

7. The emulsion of claim 1 wherein the surface-active block copolymer has a molecular weight of from 500 to 100,000 Daltons (Da).

8. The emulsion of claim 1 wherein the biodegradable encapsulating polymer comprises (poly)lactide, poly(glycolide), poly(caprolactone), a combination, or copolymer thereof.

9. The emulsion of claim 1 wherein the emulsion is substantially free of non-biodegradable polymers.

10. The emulsion of claim 1 wherein the first liquid comprises water.

11. The emulsion of claim 1 wherein the dispersed phase comprises a primary emulsion comprising a third liquid dispersed in the second liquid; wherein the third liquid and the second liquid are at least partially immiscible.

12. The emulsion of claim 1 wherein the second liquid comprises an organic solvent.

13. A microparticle comprising a biodegradable polymer matrix comprising a bioactive agent dissolved or dispersed therein and a surface-active block copolymer that comprises one or more hydrophilic blocks and one or more hydrophobic blocks; wherein the surface-active block copolymer is dissolved or dispersed in the microparticle or present on the surface of the microparticle.

14. The microparticle of claim 13 wherein the surface-active nonionic block copolymer comprises one or more hydrophilic blocks selected from poly(ethylene glycol) (PEG), poly(vinylpyrrolidone) (PVP), and polysaccharides; and one or more hydrophobic blocks selected from poly(lactide), poly(glycolide), poly(caprolactone), poly(valerolactone), poly(hydroxybutyrate), and copolymers thereof.

15. The microparticle of claim 13 wherein the one or more hydrophilic blocks of the surface-active nonionic block copolymer has a molecular weight of from 250 to 20,000 Daltons (Da).

16. The microparticle of claim 13 wherein the biodegradable surface-active block copolymer is nonionic.

17. The microparticle of claim 13 wherein the one or more hydrophobic blocks of the surface-active block copolymer has a molecular weight of from 250 to 20,000 Daltons (Da).

18. The microparticle of claim 13 wherein the surface-active block copolymer has a molecular weight of from 500 to 100,000 Daltons (Da).

19. The microparticle of claim 13 wherein the emulsion is substantially free of non-biodegradable polymers.

20. The microparticle of claim 13 wherein the biodegradable polymer matrix comprises a polymer selected from poly(lactide), poly(glycolide), poly(caprolactone), poly(valerolactone), and copolymers thereof.
